(19) United States
(12) Patent Application Publication
(10) Pub. No.: US 2026/0346030 A1
(43) Pub. Date: Nov. 5, 2026

(54) FEEDTHROUGH FLANGE INCLUDING FIXATION FEATURE FOR HEADER

(71) Applicant: Greatbatch Ltd., Clarence, NY (US)

(72) Inventors: Luis Daniel Villamil, Montevideo (UY); Mauro Ciganda, Montevideo (UY); Ignacio Augustin Armesto, Montevideo (UY)

(21) Appl. No.: 19/348,064

(22) Filed: Oct. 2, 2025

Related U.S. Application Data
(60) Provisional application No. 63/703,220, filed on Oct. 4, 2024.

(51) Int. Cl. A61N 1/375

(52) U.S. Cl. CPC A61N 1/3754

(57) ABSTRACT

In various examples, a feedthrough flange for an implantable medical device, includes a flange surface including an edge of the flange surface defining a shape complementary to a shape of an opening of a case of the implantable medical device. A fixation feature is integrally formed with the flange surface. The fixation feature is disposed proximate the edge of the feedthrough flange. The feedthrough flange is disposed within the opening and sealingly engaged with the case. The header is engaged with the feedthrough flange to couple the header to the case. The fixation feature is configured to increase engagement of the header with the feedthrough flange.

### CROSS REFERENCE TO RELATED APPLICATIONS

This application claims the benefit of priority to U.S. Provisional Application Ser. No. 63/703,220, filed on Oct. 4, 2024, entitled “FEEDTHROUGH FLANGE ANCHORAGE FIXATION FOR EPOXY HEADERS,” which is incorporated by reference herein in its entirety.

### BACKGROUND

Active implantable medical devices (AIMDs) typically have a hermetic assembly including an enclosure, housing, or case and a lid, with the electronics encapsulated inside that assembly and a connector area (called a header) where leads are connected throughout a male/female connector to deliver therapy to the proper position in the human body. These headers can be made from pre-molded parts (injected components) or, more commonly, an epoxy header casted over the connectors to create a solid polymer component.

In both cases, header delamination with respect to the hermetic assembly can be a major problem due to all the mechanical forces the hermetic assembly experiences throughout its life cycle. For instance, the hermetic assembly experiences mechanical forces during assembly and testing, during implant procedures, and while implanted. If delamination occurs, it can result in device malfunction due to loss of insulation.

To mitigate this risk, several measures are implemented during the assembly process, and specific design features are defined to enhance adhesion or bond mechanical strength. These mitigations can include a sandblasting process to increase surface roughness, validated epoxy mixing processes and curing schedules, plasma cleaning and/or standard cleaning processes, as well as chemical surface activators applied before casting or gluing the header onto the device. In terms of design, features such as mechanical bosses, either welded on or machined, are typically added to enhance header adhesion. Despite these mitigation and design features, header delamination remains a major issue in the AIMD industry, and many, if not all, regulatory bodies have expressed their concern on this topic.

Referring to FIGS. 8 and 9, conventional systems include a case 10, 10′ and/or a feedthrough flange 50, 50′ with no fixation features or with just a step 51 around the perimeter or small anchorage pins 51′ (resembling rivets) in each device corner and/or center. Referring first to FIG. 8, a conventional feedthrough flange 50 can include a step 51 around the perimeter of the flange 50 to increase the contact surface with the epoxy. Such a lid device (feedthrough flange in this case) can be welded to a deep-drawn enclosure or case 10 to close the hermetic assembly of the AIMD. Referring now to FIG. 9, a conventional feedthrough flange 50′ can include one or more anchorage pins 51′ (rivets) in one or more of the corners. These pins 51′ can be effective retainers for some header shapes and epoxy volumes but have not shown good results for all header/hermetic assembly configurations. Additionally, there are reports that, after implantation, delamination can extend beyond these steps 51 and/or rivets 51′.

### OVERVIEW

This overview is intended to provide an overview of subject matter of the present patent document. It is not intended to provide an exclusive or exhaustive explanation of the invention. The detailed description is included to provide further information about the present patent document.

The present inventors have recognized, among other things, that the present inventive subject matter can be used to maintain attachment of a header to a feedthrough flange and/or case of an implantable medical device. In some examples, the present inventive subject matter is advantageous in that it mitigates, if not eliminates, the possibility of delamination or propagation of a delamination of the header with respect to the feedthrough flange/case. In various examples, the present inventive subject matter provides for a low-cost, reliable way of attaching (and maintaining attachment of) the header of an implantable medical device to the feedthrough flange and/or case. Moreover, the present inventive subject matter is advantageous in that the form, shape, position, and/or pattern of the one or more fixation features allows for better distribution of forces at the interface between the feedthrough flange/case fixation features and header, the forces being made up of one or more of external forces, thermal variations, contraction due to epoxy curing process, or any other cause. To better illustrate the devices described herein, a non-limiting list of examples is provided here:

Example 1 can include subject matter that can include a feedthrough flange for an implantable medical device. The implantable medical device includes a header and a case, the case including an opening. A flange surface includes an edge of the flange surface defining a shape complementary to a shape of the opening of the case. A fixation feature is integrally formed with the flange surface, the fixation feature being disposed proximate the edge of the feedthrough flange. The feedthrough flange is disposed within the opening and sealingly engaged with the case. The header is engaged with the feedthrough flange to couple the header to the case. The fixation feature is configured to increase engagement of the header with the feedthrough flange.

In Example 2, the subject matter of Example 1 is optionally configured such that the fixation feature includes a raised lip extending from a top of the flange surface.

In Example 3, the subject matter of Example 1 is optionally configured such that the at least one raised lip includes a base portion extending away from the top surface of the flange surface and a lip portion extending from the base portion at an angle. The lip portion extends in a direction toward the edge of the flange surface.

In Example 4, the subject matter of any one of Examples 1-3 is optionally configured such that the fixation feature includes a groove disposed within the flange surface.

In Example 5, the subject matter of any one of Examples 1-4 is optionally configured such that the fixation feature includes at least two fixation features including at least one groove disposed within the flange surface and at least one raised lip extending from the top of the flange surface.

In Example 6, the subject matter of Example 5 is optionally configured such that the at least one raised lip includes a base portion extending away from the top surface of the flange surface and a lip portion extending from the base portion at an angle. The lip portion extends in a direction toward the edge of the flange surface.

In Example 7, the subject matter of any one of Examples 1-6 is optionally configured such that the fixation feature is integrally formed with the feedthrough flange.

In Example 8, the subject matter of any one of Examples 1-7 is optionally configured such that the fixation feature includes a plurality of fixation features disposed around a majority of the flange surface proximate the edge of the flange surface.

Example 9 can include, or can optionally be combined with any one of Examples 1-8 to include subject matter that can include an implantable medical device including a case including an opening. A header is operatively coupled to the case. A feedthrough flange includes a flange surface including an edge of the flange surface defining a shape complementary to a shape of the opening of the case. A fixation feature is integrally formed with the flange surface, the fixation feature being disposed proximate the edge of the feedthrough flange. The feedthrough flange is disposed within the opening and sealingly engaged with the case. The header is engaged with the feedthrough flange to couple the header to the case. The fixation feature is configured to increase engagement of the header with the feedthrough flange.

In Example 10, the subject matter of Example 9 is optionally configured such that the fixation feature includes a raised lip extending from a top of the flange surface.

In Example 11, the subject matter of Example 10 is optionally configured such that the at least one raised lip includes a base portion extending away from the top surface of the flange surface and a lip portion extending from the base portion at an angle. The lip portion extends in a direction toward the edge of the flange surface.

In Example 12, the subject matter of any one of Examples 9-11 is optionally configured such that the fixation feature includes a groove disposed within the flange surface.

In Example 13, the subject matter of any one of Examples 9-12 is optionally configured such that the fixation feature includes at least two fixation features including at least one groove disposed within the flange surface and at least one raised lip extending from the top of the flange surface.

In Example 14, the subject matter of Example 13 is optionally configured such that the at least one raised lip includes a base portion extending away from the top surface of the flange surface and a lip portion extending from the base portion at an angle. The lip portion extends in a direction toward the edge of the flange surface.

In Example 15, the subject matter of any one of Examples 9-14 is optionally configured such that the fixation feature is integrally formed with the feedthrough flange.

In Example 16, the subject matter of any one of Examples 9-15 is optionally configured such that the fixation feature includes a plurality of fixation features disposed around a majority of the flange surface proximate the edge of the flange surface.

Example 17 can include, or can optionally be combined with any one of Examples 1-16 to include subject matter that can include a feedthrough flange for an implantable medical device. The implantable medical device includes a header and a case. The case includes an opening. A flange surface includes an edge of the flange surface defining a shape complementary to a shape of the opening of the case. A plurality of fixation features is integrally formed with the feedthrough flange. Each of the plurality of fixation features is disposed proximate the edge of the feedthrough flange. The plurality of fixation features includes at least one groove disposed within the flange surface. At least one raised lip extends from a top of the flange surface. The feedthrough flange is disposed within the opening and sealingly engaged with the case. The header is engaged with the feedthrough flange to couple the header to the case. The plurality of fixation features is configured to increase engagement of the header with the feedthrough flange.

In Example 18, the subject matter of Example 17 is optionally configured such that the at least one raised lip includes a base portion extending away from the top surface of the flange surface and a lip portion extending from the base portion at an angle. The lip portion extends in a direction toward the edge of the flange surface.

In Example 19, the subject matter of Example 17 or Example 18 is optionally configured such that the fixation feature is machined to integrally form the fixation feature with the feedthrough flange.

In Example 20, the subject matter of any one of Examples 17-19 is optionally configured such that the fixation features are disposed around a majority of the flange surface proximate the edge of the flange surface.

### BRIEF DESCRIPTION OF THE DRAWINGS

FIG. 1 is a perspective view of an implantable medical device including fixation features in accordance with at least one example of the invention.

FIG. 2 is a side view of the implantable medical device of FIG. 1 with the header removed.

FIG. 3 is a top perspective view of fixation features of the implantable medical device of FIG. 2.

FIG. 4 is a close-up top perspective view of fixation features of FIG. 3.

FIG. 5 is a close-up top perspective view of fixation features of FIG. 3.

FIG. 6 is a cross-sectional side view of the implantable medical device of FIG. 1.

FIG. 7 is a cross-sectional end view of the implantable medical device of FIG. 1.

FIG. 8 is a top perspective view of a conventional fixation feature for an implantable medical device.

FIG. 9 is a top perspective view of a conventional fixation feature for an implantable medical device.

### DETAILED DESCRIPTION

The present inventive subject matter relates generally to an implantable medical device. More specifically, the present inventive subject matter relates to a fixation feature for an implantable medical device that improves adhesion of a header to a housing, enclosure, or case and/or feedthrough flange of the implantable medical device. In various examples, the fixation feature reduces, if not eliminates, separation and/or delamination of the header from the case and/or feedthrough flange of the implantable medical device.

The present inventive subject matter, in some examples, is directed to headers made using an epoxy casting process, where the epoxy material is cast over a hermetic assembly (for instance, a case and a feedthrough flange) using molds and core inserts and allowed to cure following a validated mixing and curing schedule. Conventional implantable medical devices can experience instances of delamination of the header occurring and potentially propagating over time to the point of losing isolative properties.

The present inventive subject matter, in some examples, includes one or more fixation features in the implantable medical device, the fixation features including specific shapes and patterns and integrated as part of the feedthrough flange. To be cost-effective, the proposed design, in some examples, is especially applicable, but not limited, to machined metallic flanges.

The present inventive subject matter, in some examples, reduces the risk of delamination, and, even if delamination occurs, the potential propagation of the non-adherence can be reduced or blocked by the proposed fixation features due to their geometry, limiting, if not preventing, delamination from spreading to the areas where the lead wires responsible for delivering energy are located inside the header of the implantable medical device (for instance, an AIMD). Since, in some examples, these features can be directly machined or added on, there can be great control over their position and mechanical strength. Consequently, in some examples, delamination can be mitigated by design, not just by process controls in the epoxy process or surface preparation.

In some examples, the present inventive subject matter defines a new form factor, profile, and/or pattern of machined anchorage features integrated into custom feedthrough flanges. These features, in some examples, can be machined as part of the flange or welded on the hermetic assembly.

In some examples, the present inventive subject matter seeks to increase the contact area between the epoxy and the hermetic assembly close to the edge that is formed between the epoxy and the hermetic assembly contact surface (since the edge is where delaminations usually start and propagate) in order to increase a maximum adhesion force that the header is able to withstand. Additionally, in some examples, various forms and patterns of these features can be used to enhance adhesion and significantly reduce, if not prevent, delamination. Such features, in some examples, add retention capabilities and distribute forces that occur at the interface between case and the header. Because the case and the header are typically formed from two different materials (for instance, titanium for the case and the feedthrough flange and epoxy polymer for the header), it is important for the hermetic assembly to be able to withstand forces caused by the different materials due to the different thermal expansion coefficients, which can generate stresses at the interface during the assembly and sterilization processes.

In some examples, the present inventive subject matter is advantageous in that the form, shape, position, and/or pattern of the fixation features allows for better distribution of forces at the interface between the feedthrough flange/case fixation features (for instance, made at least in part from titanium) and header (for instance, made from epoxy), the forces being made up of one or more of external forces, thermal variations, contraction due to epoxy curing process, or any other cause, thereby mitigating the possibility of delamination or propagation of the delamination. In a worst-case scenario, if delamination were to occur at the edge of the junction between the two parts, it will be more difficult for it to propagate beyond the defined fixation features due to their form, position, and distribution.

Conventional devices have been shown not to prevent delamination of the header in all devices and/or conditions. In some examples, the present inventive subject matter, being machined as part of the feedthrough flange, does not impact the device cost but significantly improves header adhesion. Extending the anchorage feature to all corners of the header and/or areas with higher stress concentration, without affecting cost or adding extra parts to the device, represents a significant improvement over conventional devices. In various examples, one or more fixation features can be extended around the entire perimeter of the feedthrough flange or kept just in the corners with the addition of extra grooves on the flange for enhanced adhesion.

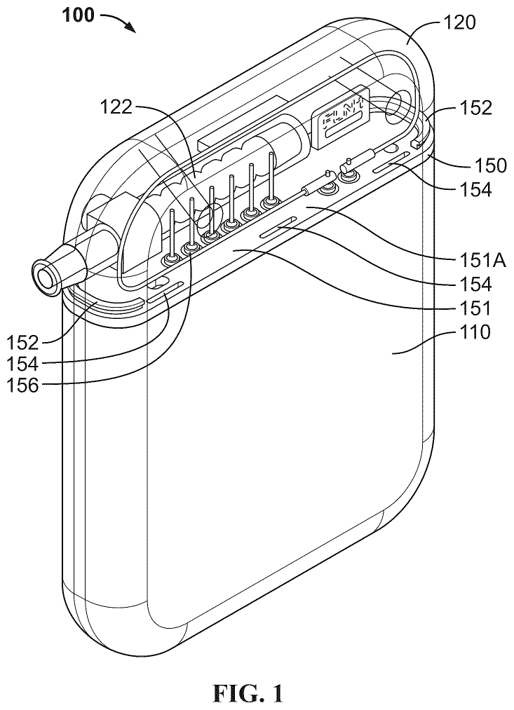

Referring now to the drawings wherein like reference numerals identify similar structural elements or features of the subject invention, there is illustrated in FIG. 1 an implantable medical device 100 configured for implantation within a patient. The implantable medical device 100, in various examples, can include various types of implantable medical devices, such as, but not limited to, a cardiac implantable pulse generator, a pacemaker, an implantable defibrillator, a neurostimulator, a cardiac monitor, a cochlear implant, etc. In various examples, the implantable medical device 100 can be implanted at various locations within the patient, including, but not limited to, a chest, a skull, a limb, a back, a pelvic region, etc. of the patient.

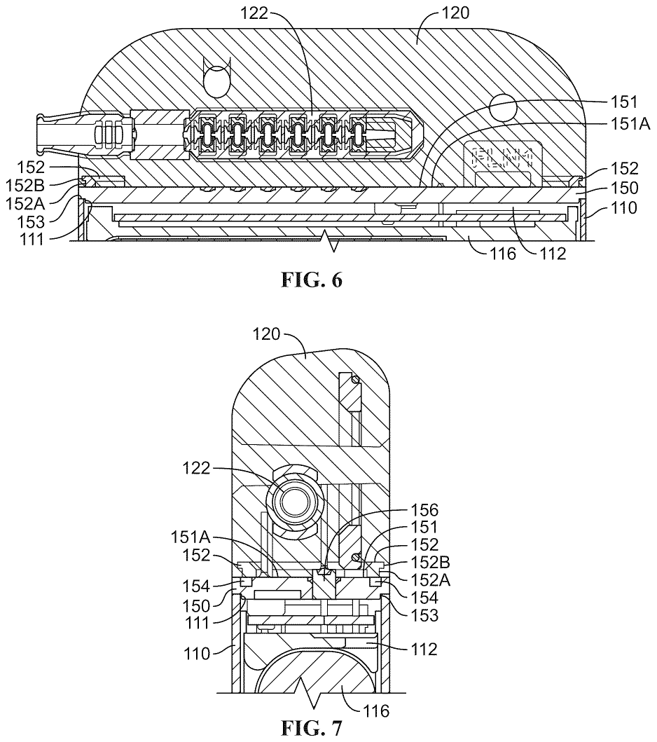

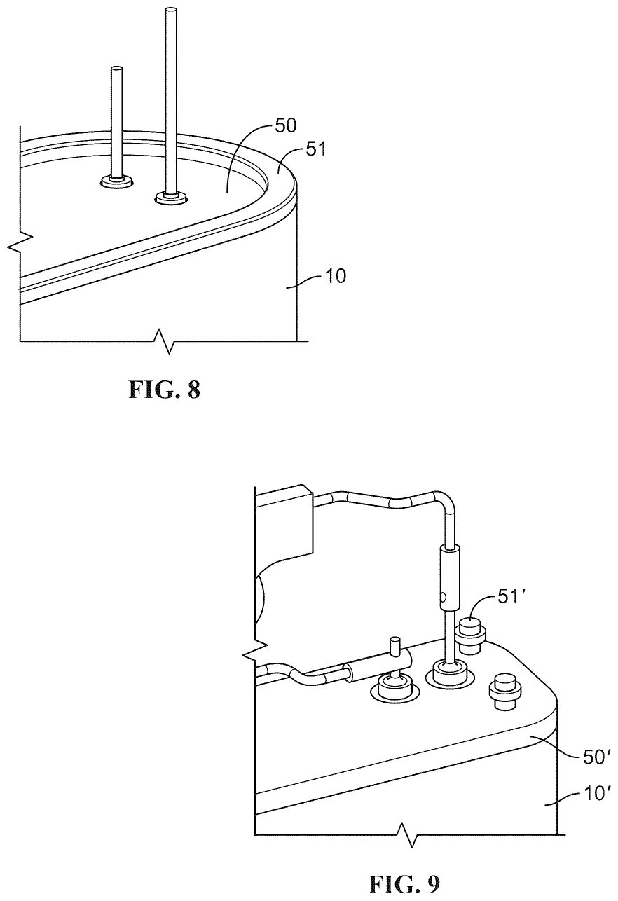

Referring now to FIGS. 1, 6, and 7, in some examples, the implantable medical device 100 includes a case 110, a feedthrough flange 150, and a header 120. In should be understood that the case 110 can be alternatively referred to as an enclosure, a housing, or other similar terms. In various examples, the case 110 of the implantable medical device 100 can be formed by stamping and/or machining. In some examples, the feedthrough flange 150 is a custom feedthrough flange 150. In some examples, the feedthrough flange 150 is disposed within an opening 111 in the case 110. In further examples, the feedthrough flange 150 is disposed within the opening 111 and sealingly engaged with the case 110. In some examples, the feedthrough flange 150 hermetically seals the opening 111 of the case 110 such that an interior 112 of the case 110 includes a hermetic environment. In some examples, the interior 112 of the case 110 includes various internal components 116 of the implantable medical device 100. In various examples, the internal components 116 include one or more of the following: one or more batteries, one or more circuit boards, one or more coils and/or antennae, one or more capacitors, one or more modules (for instance, a control module, a communication module, a sensing module, and/or the like), one or more feedthrough wires, one or more sensors, and/or the like, depending upon the application for the implantable medical device 100. For the purposes of this description, the internal components 116 are referred to in a general, collective manner.

In some examples, the header 120 is operatively coupled to the case 110. The header 120, in some examples, is engaged with the feedthrough flange 150 to couple the header 120 to the case 110. In some examples, the header 120 is formed from an epoxy material and/or another such material attached to the hermetic assembly of the case 110 and the feedthrough flange 150. In some examples, the header 120 is attached to the hermetic assembly of the case 110 and the feedthrough flange 150 with an epoxy casting process, in which the epoxy material is cast over the hermetic assembly (the case 110 and the feedthrough flange 150) using molds and core inserts and allowed to cure following a validated mixing and curing schedule. In some examples, within the header 120 are one or more of various header components 122 for the implantable medical device 100. In various examples, the header components 122 include one or more of the following: one or more stack assemblies (for accepting, for instance, a proximal end of a lead), one or more feedthrough wires, one or more identification tags, one or more coils and/or antennae, one or more electrodes, one or more sensors, and/or the like, depending upon the application for the implantable medical device 100. For the purposes of this description, the header components 122 are referred to in a general, collective manner.

In some examples, the feedthrough flange 150 includes a flange surface 151 including an edge 153 of the flange surface defining a shape complementary to a shape of the opening 111 of the case 110. In some examples, the edge 153 of the flange surface is disposed around a perimeter of the flange surface 151. In this way, in some examples, the feedthrough flange 150 can fit and be sealingly engaged within the opening 111 of the case 110. In some examples, the feedthrough flange 150 includes one or more feedthroughs 156, which function to electrically couple one or more of the internal components 116 within the interior 112 of the case 110 to one or more of the header components 122 while maintaining hermeticity within the interior 112 of the case 110.

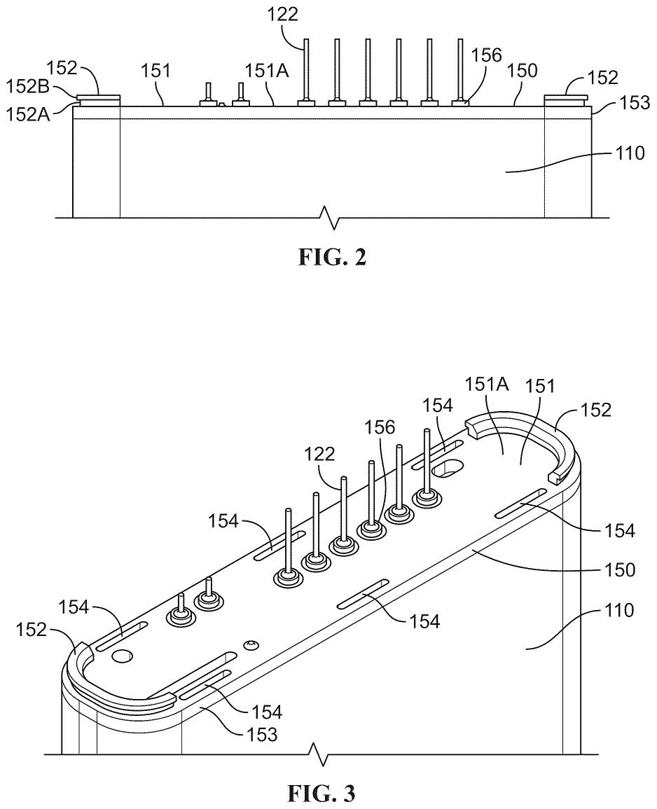

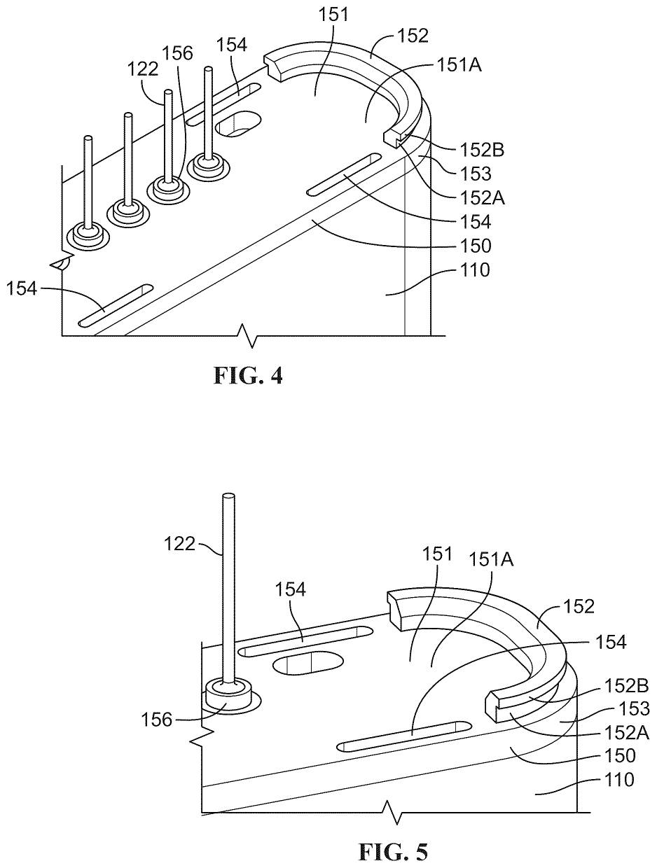

Referring now to FIGS. 1-7, in some examples, the feedthrough flange 150 includes at least one fixation feature 152, 154 integrally formed with the flange surface 151. In some examples, the fixation feature 152, 154 is disposed proximate the edge 153 of the feedthrough flange 150 and the flange surface 151. The at least one fixation feature 152, 154, in some examples, is proximate, but not immediately at, the edge 153 of the flange surface 151. That is, in some examples, the at least one fixation feature 152, 154 is spaced inwardly from the edge 153 of the flange surface 151. In some examples, the at least one fixation feature 152, 154 is configured to increase engagement of the header 120 with the feedthrough flange 150. That is, in some examples, the one or more fixation features 152, 154 are disposed at least partially around a perimeter of the feedthrough flange 150 and are configured to enhance adhesion of the header 120 to the feedthrough flange 150. In some examples, with the at least one fixation feature 152, 154 located very near the edge 153 of the flange surface 151, the at least one fixation feature 152, 154 is better configured to inhibit or otherwise limit delamination or separation of the header 120 from the feedthrough flange 150, since separation of a header from a feedthrough flange typically begins at the edge of the feedthrough flange.

In some examples, the at least one fixation feature 152 includes a raised lip 152 extending from a top 151A of the flange surface 151. In further examples, the at least one raised lip 152 includes a base portion 152A extending away from the top surface 151A of the flange surface 151 and a lip portion 152B extending from the base portion 152A at an angle. In some examples, the lip portion 152B extends in a direction toward the edge 153 of the flange surface 151. The lip portion 152B of the raised lip 152, in some examples, extends roughly perpendicularly from the base portion 152A in the direction toward the edge 153 of the flange surface 151. That is, in some examples, the at least one raised lip 152 is substantially L-shaped in cross section. In other examples, the lip portion 152B of the raised lip 152 extends from the base portion 152A at an acute angle. In still other examples, the lip portion 152B of the raised lip 152 extends from the base portion 152A at an obtuse angle. In some examples, the at least one raised lip 152 is longer than it is wide, such that the at least one raised lip 152 extends farther along the perimeter of the feedthrough flange 150 than the at least one raised lip 152 extends “radially along” (that is, generally toward a center of) the top 151A of the flange surface 151 of the feedthrough flange 150. In this way, in some examples, with forming of the header 120 in place on the feedthrough flange 150 and the case 110, the material of the header 120 flows around the at least one raised lip 152. Once cured, in some examples, the portion of the header 120 disposed around the at least one raised lip 152, and, in particular, disposed between the lip portion 152B and the top 151A of the flange surface 151, acts to anchor the header 120 with respect to the feedthrough flange 150, thereby inhibiting separation and/or delamination of the header 120 from the feedthrough flange 150.

In some examples, the at least one fixation feature 154 includes a channel or a groove 154 disposed within the flange surface 151 and the feedthrough flange 150. The at least one groove 154, in some examples, extends from the top 151A of the flange surface 151 into the flange surface 151 a distance but not far enough to extend completely through the flange surface 151. In some examples, the distance the at least one groove 154 extends into the feedthrough flange 150 can be substantially half a thickness of the feedthrough flange 150 so as to allow for adequate engagement of the header 120 within the at least one groove 154 while at the same time allowing for proper structural strength and integrity of the feedthrough flange 150. In other examples, the distance the at least one groove 154 extends into the feedthrough flange 150 can be less than half the thickness of the feedthrough flange 150 provided that amount of engagement between the at least one groove 154 and the header 120 is sufficient for the conditions within which the implantable medical device 100 is to be placed. In still other examples, the distance the at least one groove 154 extends into the feedthrough flange 150 can be more than half the thickness of the feedthrough flange 150 if more engagement between the at least one groove 154 and the header 120 is necessary for the conditions within which the implantable medical device 100 is to be placed, provided the strength and structural integrity of the feedthrough flange 150 is adequate. In this way, in some examples, with forming of the header 120 in place on the feedthrough flange 150 and the case 110, the material of the header 120 flows into the at least one groove 154. Once cured, in some examples, the portion of the header 120 disposed within the at least one groove 154 acts to anchor the header 120 with respect to the feedthrough flange 150, thereby inhibiting separation and/or delamination of the header 120 from the feedthrough flange 150.

In some examples, the at least one fixation feature 152, 154 includes at least two fixation features 152, 154, including at least one groove 154 disposed within the flange surface 151 and at least one raised lip 152 extending from the top 151A of the flange surface 151. In some examples, the at least one fixation feature 152, 154 includes a plurality of fixation features 152, 154. The at least one fixation feature 152, 154, in some examples, is disposed around a majority of the flange surface 151 proximate the edge 153 of the flange surface 151. In various examples, the plurality of fixation features 152, 154, can include all grooves 154, all raised lips 152, or a combination of grooves 154 and raised lips 152. For instance, in the example shown in FIGS. 1-7, the feedthrough flange 150 includes two raised lips 152 and six grooves 154 disposed around the flange surface 151 proximate the edge 153 of the flange surface 151. In further examples, the feedthrough flange 150 includes two raised lips 152 and six grooves 154, with one raised lip 152 extending around each of the shorter sides of the feedthrough flange 150 and three grooves 154 extending along each of the longer sides of the feedthrough flange 150 in between ends of the raised lips 152. In this way, the fixation features 152, 154 extend around a perimeter of the top 151A of the flange surface 151 to provide enhanced fixation of the header 120 to the feedthrough flange 150. In some examples, the grooves 154 are separated from neighboring grooves and/or raised lips 152 by gaps. In some examples, the fixation features 152, 154 includes at least two discrete raised lips 152 extending from the top 151A of the flange surface 151 on substantially opposite sides of the flange surface 151 from one another.

In various examples, the feedthrough flange 150 can include various configurations of one or more fixation features 152, 154 as an alternative to the configuration of fixation features 152, 154 shown in FIGS. 1-7. For instance, in some examples, the feedthrough flange 150 can include one continuous groove on each long side of the feedthrough flange 150 with raised lips 152 around each of the shorter sides of the feedthrough flange 150. In other examples, the feedthrough flange can include a single groove around the entire flange surface, the groove being disposed proximate the edge of the feedthrough flange. In further examples, the feedthrough flange can include a series of grooves around the entire flange surface, the grooves being disposed proximate the edge of the feedthrough flange and separated from neighboring grooves by gaps. In still other examples, the feedthrough flange can include a single raised lip around the entire flange surface, the raised lip being disposed proximate the edge of the feedthrough flange. In further examples, the feedthrough flange can include a series of raised lips around the entire flange surface, the raised lips being disposed proximate the edge of the feedthrough flange and separated from neighboring raised lips by gaps.

In some examples, the at least one fixation feature 152, 154 is integrally formed with the feedthrough flange 150. In further examples, the at least one fixation feature 152, 154 is integrally formed with the feedthrough flange 150 by machining the at least one fixation feature 152, 154 from the feedthrough flange 150. For instance, in some examples, the feedthrough flange 150 can be machined to form the one or more raised lips 152 extending from the flange surface 151. In some examples, the feedthrough flange 150 can be machined to form the one or more grooves 154 within the flange surface 151. In other examples, the one or more fixation features 152, 154 can be formed in the feedthrough flange 150 using 3-D printing or other additive manufacturing techniques. In still other examples, the one or more raised lips 152 can be welded or otherwise attached to the feedthrough flange 150. In still other examples, the one or more grooves 154 can be formed within the feedthrough flange 150 using cutting techniques, such as, but not limited to, plasma cutting, waterjet cutting, laser cutting, and/or oxy-fuel cutting, to name a few.

The present inventors have recognized various advantages of the subject matter described herein. The present inventors have recognized, among other things, that the present inventive subject matter can be used to maintain attachment of a header to a feedthrough flange and/or case of an implantable medical device. In some examples, the present inventive subject matter is advantageous in that it mitigates, if not eliminates, the possibility of delamination or propagation of a delamination of the header with respect to the feedthrough flange/case. In various examples, the present inventive subject matter provides for a low-cost, reliable way of attaching (and maintaining attachment of) the header of an implantable medical device to the feedthrough flange and/or case. Moreover, the present inventive subject matter is advantageous in that the form, shape, position, and/or pattern of the one or more fixation features allows for better distribution of forces at the interface between the feedthrough flange/case fixation features and header, the forces being made up of one or more of external forces, thermal variations, contraction due to epoxy curing process, or any other cause. While various advantages of the example systems are listed herein, this list is not considered to be complete, as further advantages may become apparent from the description and figures presented herein.

Although the subject matter of the present patent application has been described with reference to various examples, workers skilled in the art will recognize that changes can be made in form and detail without departing from the scope of the subject matter recited in the below claims.

The above Detailed Description includes references to the accompanying drawings, which form a part of the Detailed Description. The drawings show, by way of illustration, specific examples in which the present apparatuses and methods can be practiced. These embodiments are also referred to herein as “examples.”

The above Detailed Description is intended to be illustrative, and not restrictive. For example, the above-described examples (or one or more elements thereof) can be used in combination with each other. Other embodiments can be used, such as by one of ordinary skill in the art upon reviewing the above description. Also, various features or elements can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

In this document, the terms “a” or “an” are used to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “about” and “approximately” or similar are used to refer to an amount that is nearly, almost, or in the vicinity of being equal to a stated amount.

In the appended claims, the terms “including” and “in which” are used as the plain-English equivalents of the respective terms “comprising” and “wherein.” Also, in the following claims, the terms “including” and “comprising” are open-ended, that is, an apparatus or method that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

1. A feedthrough flange for an implantable medical device, the implantable medical device including a header and a case, the case including an opening, the feedthrough flange comprising:
a flange surface including an edge of the flange surface defining a shape complementary to a shape of the opening of the case; and
a fixation feature integrally formed with the flange surface, the fixation feature being disposed proximate the edge of the feedthrough flange;
wherein the feedthrough flange is disposed within the opening and sealingly engaged with the case, the header being engaged with the feedthrough flange to couple the header to the case, the fixation feature being configured to increase engagement of the header with the feedthrough flange.

2. The feedthrough flange of claim 1, wherein the fixation feature includes a raised lip extending from a top of the flange surface.

3. The feedthrough flange of claim 2, wherein the at least one raised lip includes a base portion extending away from the top surface of the flange surface and a lip portion extending from the base portion at an angle, the lip portion extending in a direction toward the edge of the flange surface.

4. The feedthrough flange of claim 1, wherein the fixation feature includes a groove disposed within the flange surface.

5. The feedthrough flange of claim 1, wherein the fixation feature includes at least two fixation features including at least one groove disposed within the flange surface and at least one raised lip extending from the top of the flange surface.

6. The feedthrough flange of claim 5, wherein the at least one raised lip includes a base portion extending away from the top surface of the flange surface and a lip portion extending 16 from the base portion at an angle, the lip portion extending in a direction toward the edge of the flange surface.

7. The feedthrough flange of claim 1, wherein the fixation feature is integrally formed with the feedthrough flange.

8. The feedthrough flange of claim 1, wherein the fixation feature includes a plurality of fixation features disposed around a majority of the flange surface proximate the edge of the flange surface.

9. An implantable medical device comprising:
a case including an opening;
a header operatively coupled to the case; and
a feedthrough flange including:
a flange surface including an edge of the flange surface defining a shape complementary to a shape of the opening of the case; and
a fixation feature integrally formed with the flange surface, the fixation feature being disposed proximate the edge of the feedthrough flange;
wherein the feedthrough flange is disposed within the opening and sealingly engaged with the case, the header being engaged with the feedthrough flange to couple the header to the case, the fixation feature being configured to increase engagement of the header with the feedthrough flange.

10. The implantable medical device of claim 9, wherein the fixation feature includes a raised lip extending from a top of the flange surface.

11. The implantable medical device of claim 10, wherein the at least one raised lip includes a base portion extending away from the top surface of the flange surface and a lip portion extending from the base portion at an angle, the lip portion extending in a direction toward the edge of the flange surface. 17

12. The implantable medical device of claim 9, wherein the fixation feature includes a groove disposed within the flange surface.

13. The implantable medical device of claim 9, wherein the fixation feature includes at least two fixation features including at least one groove disposed within the flange surface and at least one raised lip extending from the top of the flange surface.

14. The implantable medical device of claim 13, wherein the at least one raised lip includes a base portion extending away from the top surface of the flange surface and a lip portion extending from the base portion at an angle, the lip portion extending in a direction toward the edge of the flange surface.

15. The implantable medical device of claim 9, wherein the fixation feature is integrally formed with the feedthrough flange.

16. The implantable medical device of claim 9, wherein the fixation feature includes a plurality of fixation features disposed around a majority of the flange surface proximate the edge of the flange surface.

17. A feedthrough flange for an implantable medical device, the implantable medical device including a header and a case, the case including an opening, the feedthrough flange comprising:
a flange surface including an edge of the flange surface defining a shape complementary to a shape of the opening of the case; and
a plurality of fixation features integrally formed with the feedthrough flange, each of the plurality of fixation features being disposed proximate the edge of the feedthrough flange, the plurality of fixation features including:
at least one groove disposed within the flange surface, and at least one raised lip extending from a top of the flange surface;
wherein the feedthrough flange is disposed within the opening and sealingly engaged with the case, the header being engaged with the feedthrough flange to couple the header to the case, the plurality of fixation features being configured to increase engagement of the header with the feedthrough flange.

18. The feedthrough flange of claim 17, wherein the at least one raised lip includes a base portion extending away from the top surface of the flange surface and a lip portion extending from the base portion at an angle, the lip portion extending in a direction toward the edge of the flange surface.

19. The feedthrough flange of claim 17, wherein the fixation feature is machined to integrally form the fixation feature with the feedthrough flange.

20. The feedthrough flange of claim 17, wherein the fixation features are disposed around a majority of the flange surface proximate the edge of the flange surface.
